(19) United States
(12) Patent Application Publication
(10) Pub. No.: US 2003/0215917 A1
(43) Pub. Date: Nov. 20, 2003

(54) Assay for evaluation of activity of compounds against HCV using a novel detection system in the HCV replicon

(76) Inventors: Mingjun Huang, Potomac, MD (US); Yongnian Sun, Hamden, CT (US); Wengang Yang, Cheshire, CT (US); Yongsen Zhao, Branford, CT (US); Joanne Fabrycki, Southington, CT (US)

Correspondence Address: HALE AND DORR, LLP, 60 STATE STREET, BOSTON, MA, 02109

(21) Appl. No.: 10/407,066

(22) Filed: Apr. 3, 2003

Related U.S. Application Data
(60) Provisional application No. 60/369,923, filed on Apr. 4, 2002.

(51) Int. Cl. C12Q 1/70; C12P 21/06; C07H 21/04; C07K 1/00; C07K 14/00; C07K 17/00

(52) U.S. Cl. 435/69.1; 435/5; 530/350; 536/23.72

(57) ABSTRACT

The present invention relates to a cell-based HTS assay for evaluation of antiviral activity of compounds against HCV using HCV replicon.

### CROSS REFERENCE TO RELATED APPLICATIONS

This application claims priority to U.S. Provisional Application No. 60/369,923 filed Apr. 4, 2002.

### FIELD OF THE INVENTION

The present invention relates to compositions and methods for the quantification of the extent of replication of the Hepatitis C viral genome in an in vitro tissue culture system. In particular, the present invention relates to a replicon assay system for identifying novel drug substances in a high throughput screening format.

### BACKGROUND OF THE INVENTION

Hepatitis is a disease occurring throughout the world. It is generally of viral nature, although there are other causes known. Viral hepatitis is by far the most common form of hepatitis. Viral hepatitis is known to be caused by five different viruses known as hepatitis A,B,C, D and E. Hepatitis A virus (HAV) is an RNA virus and does not lead to long-term clinical symptoms. Hepatitis B virus (HBV) is a DNA virus. Hepatitis D virus (HDV) is a dependent virus that is unable to infect cells in the absence of HBV. Hepatitis E virus (HEV) is a water-borne virus. Hepatitis C virus (HCV) was first identified and characterized as a cause of non-A, non-B hepatitis (NANBH). Houghton et al., EPO Pub. No. 388,232. This led to the disclosure of a number of general and specific polypeptides useful as immunological reagents in identifying HCV. See, e. g., Choo et al. (1989) Science, 244:359-362; Kuo et al. (1989) Science, 244:362-364; and Houghton et al. (1991) Hepatology, 14:381-388. HCV is the major cause of blood transfusion-related hepatitis. An estimated 170 million people worldwide have been infected by HCV—a number more than four times as many as HIV (Cohen, 1999). In the United States, antibodies to HCV are detected in approximately 1˜2.4% of the general population. In other words, approximately 4 million people in the United States are infected with HCV (CDC, 1998). The acute phase of HCV infection is usually associated with mild symptoms (Aach et al., 1991; Alter et al, 1991; Koretz et al., 1993). However, evidence has accumulated that suggests that only 15%˜20% of the infected people will clear HCV from the bloodstream, leaving 75˜85% to develop into a long-term chronic infection status (Alter et al., 1992; Esteban et al.,1991; Seeff et al., 1992; Shakil et al., 1995). Among this group of chronically infected people, 10˜20% will progress to life-threatening conditions known as cirrhosis and another 1˜5% will develop a liver cancer called hepatocellular carcinoma (Di et al., 1991a; Di et al., 1991b; Fattovich et al., 1997; Kiyosawa et al., 1990; Seeff et al., 1992). Unfortunately, the entire infected population is at risk for these life-threatening conditions because no one can predict which individual will eventually progress to any of them. HCV appears in the blood of infected individuals at very low rates relative to other infectious viruses, making the virus very difficult to detect.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post- transfusion has been markedly reduced lately due to advances in diagnostic tools used to screen blood donors.

Although the need for an effective vaccine is great, its development is unlikely in the near future because of the lack of efficient cell culture systems and small animal models; the presence of a weak neutralizing humoral and protective cellular immune response; and the marked genetic variability of the virus.

Modest progress has been made in the past several years for HCV chemotherapy. The only FDA approved treatments currently available for HCV infection are interferon-α(IFN-α) monotherapy, or interferon and ribavirin combination therapy. A pegylated version of interferon offers some advantages of increased freedom from side effects. However, many patients respond poorly. According to different clinical studies, only 70% of treated patients normalize alanine aminotransferase (ALT) levels in the serum and after discontinuation of IFN, 35% to 45% of these responders relapse. Even with the use of pegylated interferon and ribavirin, the sustained response rate is only about 30˜40% in HCV type-1 patients (Di et al., 2002). Therefore, as promising as the combination therapy may be there is a great need for the further development of anti-viral agents.

HCV is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. HCV particles have been isolated from pooled human plasma and shown, by electron microscopy, to have a diameter of about 50-60 nm. The HCV genome is a single-stranded, positive-sense RNA of about 9,600 base pairs coding for a polyprotein of 3009-3030 amino-acids, which is cleaved co- and post-translationally by cellular and two viral proteinases into mature viral proteins (core, E1, E2, p7, NS2, NS3, NS4A, NS4B, NS5A, NS5B). It is believed that the structural proteins, E1 and E2, the major glycoproteins are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

At least six major genotypes and more than 80 subtypes of HCV have been identified based on extensive sequence comparison of either the complete HCV genome or the HCV core, envelope 1, NS 5, 5′ noncoding and NS2 region (Buck et al., 1995; Simmonds 1995). The major genotypes show approximately 65% homology overall, and related subtypes show 77% to 79% homology. Different genotypes have been shown to affect disease severity and virus-host interactions. The most common genotypes in the United States and Western Europe are 1a and 1b. Genetic variability may also contribute to the spectrum of different responses observed after IFN-a treatment of chronically infected patients.

An HCV replicon system (Lohmann et al., 1999) has been used widely to discover new antiviral agents. The conventional readout methods for assaying the inhibitory effect of compounds on HCV using the replicon system, e.g., Northern Blot analysis, Dot blot analysis or Real time PCR, require that the RNA be purified, which is time consuming, expensive, and error-prone. Historically, these limitations have made high throughput screening impractical, if not impossible.

Numerous attempts have been made by many different investigators to infect mammalian cells in vitro with serum collected from HCV-infected individuals, and low levels of replication have been reported in a number of cells types infected by this method [Bertolini et al., Res. Virol. 144: 281-285 (1993); Kato et al., Biochem. Biophys. Res. Commun. 206:863-9 (1996); Mizutani et al., J. Virol. 70: 7219-7223 (1996); Cribier et al., J. Gen. Virol., 76: 2485-2491 (1995)]. Although the level of replication is low, long-term infections can occur. However, efficient HCV replication has not been observed in any of the cell-culture systems described to date.

An HCV replicon system has recently been developed (Lohmann, et. al., 1999) which allows the quantification of viral genomic transcription in a model system. This HCV replicon (FIG. 1) contains non-structural viral genome in a stably transfected human hepatoma cell line (Huh7) and can be used to partially represent the replication, transcription and translation of HCV. This HCV replicon system is the generally accepted model system for studying HCV replication.

There remains a need for a high throughput screening format detection system with improved sensitivity and detection limits which can be used with the replicon system.

### SUMMARY OF THE INVENTION

The invention provides a novel high throughput format assay method for analysis of the antiviral and cell toxicity activity of test compounds upon Hepatitis C virus. The method includes: (a) constructing a hepatitis C replicon system containing a gene required for said replicon to replicate; (b) transfecting and propagating cells which contain said replicon; (c) selecting stably transfected clones; (d) growing and propagating said stably transfected clones; (e) plating cells and adding test compounds to wells containing cells transfected with said replicon; (f) allowing the transfected cells to incubate in the presence of the test compound(s); (g) at the end of the incubation period, quantifying the gene product protein produced by the replicon using a measurement system, e.g., biological assay, immunoassay, radioimmunoassay, or by calorimetric, fluorogenic, or chemiluminescent assay; and (h) at the end of the incubation period quantifying the marker for cellular protein expression level produced by the cells to determine cell number as a measure of test compound toxicity.

The amount of gene product protein produced during the incubation period can be quantified by any standard assay such as, for example, using a luminescence assay, a chemiluminesence assay, an enzyme-multiplied immunoassay technology (EMIT) assay, a fluorescence resonance excitation transfer immunoassay (FRET) assay, an enzyme channeling immunoassay (ECIA) assay, a substrate-labeled fluorescent immunoassay (SLFIA) assay, a fluorescence polarization assay, a fluorescence protection assay, an antigen-labeled fluorescence protection assay (ALFPIA), or scintillation proximity assay (SPA). Methods for measuring the amount of the marker for cellular protein expression level include, but are not limited to, visual inspection and an MTS uptake assay.

Genes required for the HCV replicon to replicate include, but are not limited to, NPTII, hygromycin B, Puromycin, HCV Ns2 protein, HCV Ns3 protein, HCV Ns4a protein, HCV Ns4b protein, HCV Ns5a protein and HCV Ns5b protein. Markers for cellular protein expression level include, but are not limited to, albumin and GADPH. In one embodiment, markers have an intracellular half-life of between 0.8 hours and eight hours. In certain embodiments, the intracellular half-life of the marker is between one and four hours.

The invention also includes a novel high throughput format assay method for analysis of the antiviral and cell toxicity activity of test compound(s) upon Hepatitis C virus. The method includes: (a) constructing a Hepatitis C replicon system containing neomycin phosphotransferase II (NPT) gene (replicon); (b) transfecting a propagating cells which contain said replicon; (c) selecting stably transfected clones; (d) growing and propagating said stably transfected clones; (e) plating cells and adding test compounds to wells containing cells transfected with said replicon; (f) allowing said transfected cells to incubate in the presence of the test compound(s); (g) quantifying the NPT produced by said replicon during the incubation period as a measure of antiviral activity of said test compound(s); and (h) quantifying albumin produced by the cells during the incubation period to determine cell number as a measure of toxicity of said test compound(s).

The amount of NPT produced during the incubation period can be measured by any standard assay such as, for example, using a luminescence assay, a chemiluminescence assay, an enzyme-multiplied immunoassay technology (EMIT) assay, a fluorescence resonance excitation transfer immunoassay (FRET) assay, an enzyme channeling immunoassay (ECIA) assay, a substrate-labeled fluorescent immunoassay (SLFIA) assay, a fluorescence polarization assay, a fluorescence protection assay, an antigen-labeled fluorescence protection assay (ALFPIA), or scintillation proximity assay (SPA).

The amount of albumin produced during the incubation period can be measured by any standard assay such as, for example, using a luminescence assay, a chemiluminescence assay, an enzyme-multiplied immunoassay technology (EMIT) assay, a fluorescence resonance excitation transfer immunoassay (FRET) assay, an enzyme channeling immunoassay (ECIA) assay, a substrate-labeled fluorescent immunoassay (SLFIA) assay, a fluorescence polarization assay, a fluorescence protection assay, an antigen-labeled fluorescence protection assay (ALFPIA), or scintillation proximity assay (SPA).

The invention also includes kits suitable for use in the novel detection system. Such kits include polypeptides, epitopes or antibodies in suitable containers, along with reagents and materials required for the conduct of the assay, as well as assay instructions.

The invention also includes a novel high throughput format assay method for simultaneous analysis of the antiviral and cell toxicity activity upon Hepatitis C virus of test compound(s). The method includes: (a) constructing a Hepatitis C replicon system containing a gene required for the replicon to replicate; (b) transfecting and propagating cells which contain the replicon; (c) selecting stably transfected clones; (d) plating the cells and adding test compound(s) to wells containing the cells transfected with the replicon; (e) allowing the cells to incubate in the presence of the test compound(s); simultaneously quantifying the amount of the gene product protein produced by the replicon during the incubation period, and the amount of the marker for cellular protein expression level produced by the cells during the incubation period.

Numerous additional aspects and advantages of the invention will be apparent to those skilled in the art upon consideration of the following detailed description of the invention.

### BRIEF DESCRIPTION OF THE DRAWINGS

FIG. 1 is a schematic representation of the genome structure of HCV replicon.

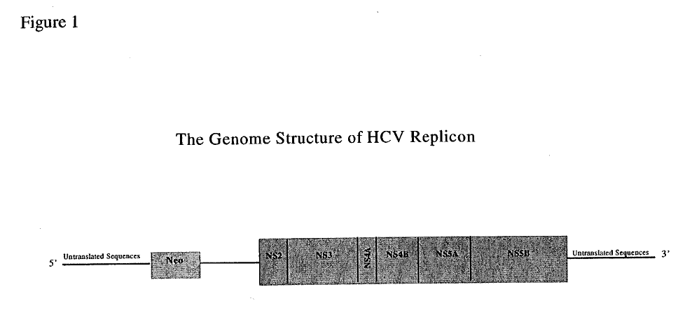

FIG. 2 is a schematic representation of the assay for evaluation of activity of compounds against HCV using HCV replicon.

FIG. 3 is a graphical representation of the NPT level of HCV replicon containing cells after treatment with various concentrations of interferon alpha.

FIG. 4 is a graphical representation of the detection of albumin in HCV replicon cells treated with helioxanthin.

FIG. 5 is a graphical representation of the detection of cytotoxicity of HCV replicon cells treated with helioxanthin MTS reagent (Promega G3580) according to manufacturer's instructions.

### DETAILED DESCRIPTION OF THE INVENTION

The invention provides a method for a cell-based HTS assay for evaluation of antiviral activity of compounds against HCV using HCV replicon.

The method of the invention includes a novel HTS assay system, in which the efficacy of a test compound upon the replication of the HCV replicon as well as the toxicity of the test compound to normal cells can be measured in the same well of a standard assay plate.

In one embodiment of the invention, the effect of test compounds upon the replication in the replicon test system is quantified with the aid of the enzyme neomycin phosphotransferase II (NPT) product which is integrated into the replicon genome. To measure this, the albumin or the gene protein of a surrogate marker of the HCV replicon cells is also utilized with the aid of an immuno- or other assay. The quantity of cellular protein albumin in HCV replicon cell lysate correlates well with the number of cells. Therefore, it can be used to monitor the cell number (to represent the level of cytotoxicity) and protein level to provide a normalization reference for antiviral activity of compounds. Neomycin phosphotransferase II gene (Neo) is selected as the target of detection because as an integrated part of the HCV replicon genome, the expression of NPT is under the similar viral regulation to that of HCV replicon.

The novel HTS assay system of the invention allows a simple and cost efficient way to achieve high throughput compound screening with clear advantages over all currently used methods to detect HCV RNA. Current methods all require purification of RNA which is time consuming, expensive and error prone. Anytime pre-PCR purification is performed, the risk of contamination is increased. In spite of these problems, real time PCR is currently a popular method for detecting HCV RNA.

The concept of combining amplification with product analysis has become known as real time PCR. See, for example, WO/9746707A2, WO/9746712A2, WO/9746714A1, all published Dec. 11, 1997. Monitoring fluorescence of each cycle of PCR initially involved the use of ethidium bromide. Other fluorescent systems (“molecular beacons”) have been developed that are capable of providing additional data concerning the nucleic acid concentration and sequence. Chemiluminescent systems can also be used, instead of fluorescent reporters although it is unclear if they present any fundamental advantage, see for example WO0173129 A2.

Unfortunately, the practical implementation of real time PCR techniques has lagged behind the conceptual promise. Currently available instrumentation does not actually analyze data during PCR; it simply acquires the data for later analysis. After PCR has been completed, multiple manual steps are necessary to analyze the acquired data, and human judgment is typically required to provide the analysis result. A major problem in automating PCR data analysis is identification of baseline fluorescence. Background fluorescence varies from reaction to reaction. Moreover, baseline drift, wherein fluorescence increases or decreases without relation to amplification of nucleic acids in the sample, is a common occurrence.

A “replicon” as used herein includes any genetic element, for example, a plasmid, cosmid, bacmid, phage or virus, that is capable of replication largely under its own control. A replicon may be either RNA or DNA and may be single or double stranded.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule can be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The term “sequence similarity” in all its grammatical forms refers to the degree of identity or correspondence between nucleic acid or amino acid sequences of proteins that may or may not share a common evolutionary origin. However, in common usage and in the instant application, the term “homologous,” when modified with an adverb such as “substantially” or “highly,” refers to sequence similarity and not a common evolutionary origin.

In a specific embodiment, two DNA or RNA sequences are “homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. 1 & 11, supra; Nucleic Acid Hybridization, supra.

It should be appreciated that the terms HCV sequence, such as the “3′ terminal sequence element, “3′ terminus,” 3′ sequence element, “are meant to encompass all of the following sequences: (i) an RNA sequence of the positive-sense genome RNA; (ii) the complement of this RNA sequence, i.e., the HCV negative-sense RNA; (iii) the DNA sequence corresponding to the positive-sense sequence of the RNA element; and (iv) the DNA sequence corresponding to the negative-sense sequence of the RNA element.

Similarly, in a particular embodiment, two amino acid sequences are “homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program. Most specifically, the terms “percent similarity”, “percent identity” and “percent homology” when referring to a particular sequence are used as set forth in the GCG software program.

A “fragment” or “portion” of the HCV genome refers to a sequence, when translated as a polypeptide comprising a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids.

A “derivative” of a HCV genome polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential biological activity of the original material.

Different “variants” of the HCV genome exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia:

a) variants in which one or more amino acids residues are substituted with conservative or non-conservative amino acids

b) variants in which one or more amino acids are added

c) variants in which one or more amino acids include a substituent group,

The term “functional” as used herein means that the nucleic or amino acid sequence is functional for the recited assay or purpose.

A “vector” is a replicon, such as a plasmid, cosmid, bacmid, phage or virus, to which another genetic sequence or element (either DNA or RNA) may be attached so as to bring about the replication of the attached sequence or element.

A “cassette” refers to a segment of DNA or RNA that can be inserted into a vector at specific restriction sites. The segment of DNA or RNA encodes a polypeptide or RNA of interest, and the cassette and restriction sites are designed to ensure insertion of the cassette in the proper reading frame for transcription and translation

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

A “promoter sequence” is a DNA or RNA regulatory region capable of binding RNA polymerase in a cell and initiating transcription of a downstream (3′ direction) coding or noncoding sequence. Thus, promoter sequences can also be used to refer to analogous RNA sequences or structures of similar function in RNA virus replication and transcription.

Preferred promoters for cell-free or bacterial expression of infectious HCV DNA clones of the invention are the phage promoters T7, T3, and SP6. Alternatively, a nuclear promoter, such as cytomegalovirus immediate-early promoter, can be used. Indeed, depending on the system used, expression may be driven from a eukaryotic, prokaryotic, or viral promoter element. Promoters for expression of HCV RNA can provide for capped or uncapped transcripts.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions.

Therefore, the probe sequence need not reflect the exact complementary sequence of the target. For example, a 16 non-complementary nucleotide fragment may be attached to the 51 or 31 end of the probe, with the remainder of the probe sequence being complementary to the target strand.

Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography.

Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

As used herein, the term “marker” shall mean an operative genetic system in which a nucleic acid comprises a gene that encodes a product that when expressed produces a signal that is a readily measurable, e.g., by biological assay, immunoassay, radioimmunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the gene product. The required control elements will vary according to the nature of the and whether the gene is in the form of DNA or RNA, but may include, but not be limited to, such elements as promoters, enhancers, translational control sequences, poly A addition signals, transcriptional termination signals and the like.

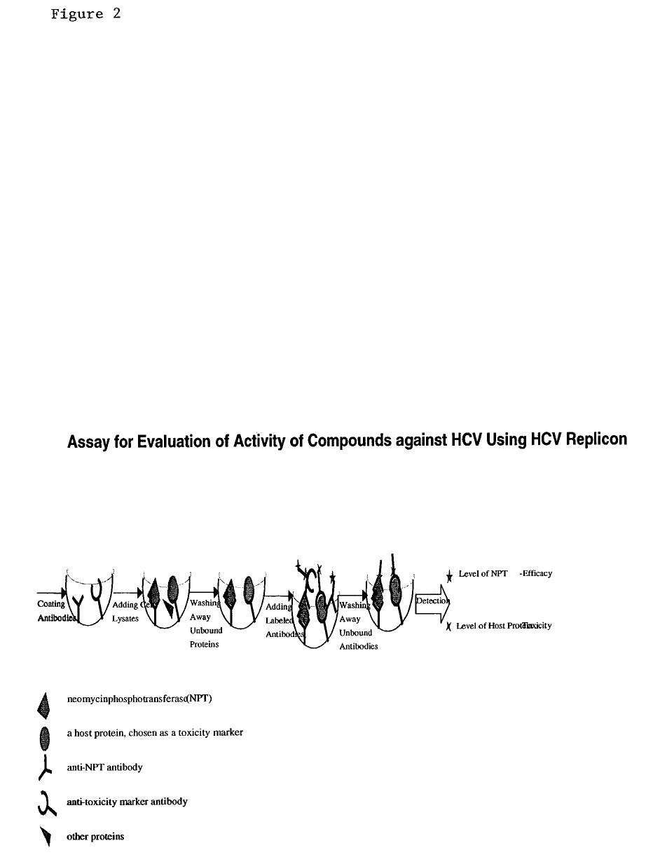

A cell-based HTS assay for evaluation of antiviral activity of compounds against HCV using HCV replicon has been developed. This novel assay system enables measurement of the efficacy and toxicity in the same well of a standard well assay plate (FIG. 2). In one embodiment, neomycin phosphotransferase II (NPT) and albumin of HCV replicon cells was measured using ELISA. Neomycin phosphotransferase II gene (Neo) was selected as the target of detection because as an integrated part of the HCV replicon genome, the expression of NPT is under the similar viral regulation to that of HCV replicon. Furthermore, the quantity of cellular protein albumin in HCV replicon cell lysate correlates well with the number of cells. Therefore, it can be used to monitor the cell number (to represent the level of cytotoxicity) and protein level to provide a normalization reference for the antiviral activity of compounds.

Suitable host cells for the present invention are mammalian cells. In an embodiment, host cells are derived from human tissues and cells which are the principle targets of viral infection. These include but are not limited to human cells such as hepatoma cell lines (HepG2, Huh 7), primary human hepatocytes, human T cells, monocytes, macrophage, dendritic cells, Langerhans cells, hematopoeitic stem cells and precursor cells.

The measurement step can be performed by many methods. Generally, immunoassays fall into the following two categories. First, antibody-antigen precipitation tests, such as radial immunodiffusion, hemagglutination, and coated latex particle agglutination. Second, labeled-reagent tests, such as radioimmunoassay, and enzyme-linked immunoassay. The precipitation type tests have the advantage of being performed manually and are commercially used in disposable kits which are read visually and do not require an instrument. The reading from a precipitation type immunoassay is usually expressed as the presence or absence of an agglutination reaction at each of a series of known dilutions of the test sample or competing antigen. The disadvantages of the precipitation type tests is that they are much less sensitive than the labeled reagent assays, require time consuming incubation steps, and are susceptible to subjective error in visual identification of a precipitation reaction.

Labeled reagent immunoassays are quantitative and highly sensitive but, nevertheless, have certain disadvantages. Radioimmunoassays employ radioactive tracers and, therefore, require a gamma radiation detection instrument. The radioactive tracers have a short shelf life, pose a health hazard to the technician and have been subject to restrictive legislation. Enzyme-linked immunoabsorbant assays (ELISA) use reagents labeled with an enzyme. The enzyme is detected by its reaction with a substrate to yield a product that can be easily measured (for example by formation of a color). The ELISA does not require radioactive materials and uses reagents with a long shelf life.

The ELISA assay begins with the binding of a reference reagent to a solid phase support, such as the bottom of a plastic well. Test fluid, mixed with enzyme-labeled reagent, is reacted with the bound reference reagent. Through a number of dilution, incubation and washing steps (as many as fourteen), bound and free reagents are separated, and a color forming reaction is initiated. The intensity of the color formed at different serial dilutions provides the quantitative measure.

Various other assays may alternatively be used to determine the amount of albumin or the gene product protein of the surrogate marker, and various assays may be also used to determine the level of expressed neomycin phosphotransferase.

Design of the immunoassays is subject to a great deal of variation, and many formats are known in the art. An immunoassay may use, for example, a monoclonal antibody directed towards a given epitope(s), a combination of monoclonal antibodies directed towards epitopes of a single antigen, monoclonal antibodies directed towards multiple different antigens, polyclonal antibodies directed towards the same antigen, or polyclonal antibodies directed towards different antigens. Protocols may be based, for example, upon competition, or direct reaction, or sandwich type assays. Protocols may also, for example, use solid supports, or may be by immunoprecipitation. Most assays involve the use of labeled antibody or polypeptide; the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Luminescence is the production of light by any means, including photoexcitation or a chemical reaction. Chemiluminescence is the emission of light only by means of a chemical reaction. It can be further defined as the emission of light during the reversion to the ground state of electronically excited products of chemical reactions (Woodhead, J. S. et al., Complementary Immunoassays, W. P. Collins ed., (John Wiley & Sons Ltd.), pp. 181-191 (1988)). Chemiluminescent reactions can be divided into enzyme-mediated and nonenzymatic reactions. It has been known for some time that the luminescent reactant luminol can be oxidized in neutral to alkaline conditions (pH 7.0-10.2) in the presence of oxidoreductase enzymes (horseradish peroxidase, xanthine oxidase, glucose oxidase), H₂O₂, certain inorganic metal ion catalysts or molecules (iron, manganese, copper, zinc), and chelating agents, and that this oxidation leads to the production of an excited intermediate (3-aminophthalic acid) which emits light on decay to its ground state.

Typically, an immunoassay will involve selecting and preparing the test sample suspected of containing the antibodies, such as a biological sample, then incubating it with an antigenic polypeptide(s) under conditions that allow antigen-antibody complexes to form, and then detecting the formation of such complexes. Suitable incubation conditions are well known in the art. The immunoassay may be, without limitations, in a heterogeneous or in a homogeneous format, and of a standard or competitive type. In a heterogeneous format, the polypeptide is typically bound to a solid support to facilitate separation of the sample from the polypeptide after incubation.

The amount or extent of binding to the solid support which is desirable varies depending upon the identity and concentration of the particular species involved. Any of the polymeric materials described in U.S. Pat. No. 3,646,346 may be used.

Commonly, the solid support consists of nitrocellulose. Nitrocellulose is cheap, simple to use and has a long shelf life. However, any solid support may be used. A heat bonding of the nitrocellulose support to a plastic backing may also be used as is described in EP0324603.

Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride (known as Immulon.™), diazotized paper, nylon membranes, activated beads, and Protein A beads. For example, Dynatech Immulon™ or Immulon™ microtiter plates or 0.25-inch polystyrene beads (Precision Plastic Ball) can be used in the heterogeneous format. The solid support containing the antigenic polypeptide is typically washed after separating it from the test sample, and prior to detection of bound antibodies. Both standard and competitive formats are known in the art.

In a homogeneous format, the test sample is incubated with antigen in solution. For example, it may be under conditions that will precipitate any antigen-antibody complexes which are formed. Both standard and competitive formats for these assays are known in the art.

The homogeneous assay, which is known under the designation EMIT (enzyme-multiplied immunoassay technology) (Biochem. Biophys. Res. Commun. 47: 846, 1972), has proved to be of value for detecting small molecules, for example of drugs (e.g. steroids). In a modified EMIT, the activity of the enzyme being used as label decreases when the analyte/enzyme conjugate binds to the antibody which is directed against the analyte. This is apparently due to a diminished affinity of the substrate for the active center of the enzyme in the presence of the antibody, or to steric hindrance, or to a conformational change in the enzyme.

A further variant of EMIT is based on inhibition of the enzymic activity by the analyte derivative which is bound covalently to the enzyme. In this case, the activity is restored when the antibody, which is directed against the analyte, binds to the enzyme-labeled analyte derivative. Another variant of this method has been developed for relatively large analytes such as, for example, IgG (Anal. Biochem. 102: 167, 1990). However, the sensitivity which is achieved using this method is fairly low.

FRET (fluorescence resonance excitation transfer immunoassay; J. Biol. Chem. 251: 4172, 1976) is based on the transfer of energy between two fluorescent molecules, one of which is linked to the antibody while the other is linked to the analyte derivative. In this case, the analyte which is to be detected prevents formation of the complex between the labeled antibody and the labeled analyte derivative. FRET is detectable when two fluorescent labels which fluoresce at different frequencies are sufficiently close to each other that energy is able to be transferred from one label to the other. FRET is widely known in the art (for a review, see Matyus, 1992, J. Photochem. Photobiol. B: Biol., 12: 323-337, which is herein incorporated by reference).

FRET is a radiationless process in which energy is transferred from an excited donor molecule to an acceptor molecule. The efficiency of this transfer is dependent upon the distance between the donor an acceptor molecules, as described below. Since the rate of energy transfer is inversely proportional to the sixth power of the distance between the energy donor and acceptor, the energy transfer efficiency is extremely sensitive to distance changes. Energy transfer is said to occur with detectable efficiency in the 1-10 nm distance range, but is typically 4-6 nm for favorable pairs of donor and acceptor. Radiationless energy transfer is based on the biophysical properties of fluorophore.

These principles are reviewed elsewhere (Lakowicz, 1983, Principles of Fluorescence Spectroscopy, Plenum Press, New York; Jovin and Jovin, 1989, Cell Structure and Function by Microspectrofluorometry, eds. E. Kohen and J. G. Hirschberg, Academic Press). Briefly, a fluorophore absorbs light energy at a characteristic wavelength. This wavelength is also known as the excitation wavelength. The energy absorbed by a fluorochrome is subsequently released through various pathways, one being emission of photons to produce fluorescence. The wavelength of light being emitted is known as the emission wavelength and is an inherent characteristic of a particular fluorophore. Radiationless energy transfer is the quantum-mechanical process by which the energy of the excited state of one fluorophore is transferred without actual photon emission to a second fluorophore. That energy may then be subsequently released at the emission wavelength of the second fluorophore. The first fluorophore is generally termed the donor (D) and has an excited state of higher energy than that of the second fluorophore, termed the acceptor (A). The essential features of the process are that the emission spectrum of the donor overlaps with the excitation spectrum of the acceptor, and that the donor and acceptor be sufficiently close.

The distance over which radiationless energy transfer is effective depends on many factors including the fluorescence quantum efficiency of the donor, the extinction coefficient of the acceptor, the degree of overlap of their respective spectra, the refractive index of the medium, and the relative orientation of the transition moments of the two fluorophores. In addition to having an optimum emission range overlapping the excitation wavelength of the other fluorophore, the distance between D and A must be sufficiently small to allow the radiationless transfer of energy between the fluorophores.

FRET may be performed using proteins labeled by methods known in the art, using a fluorimeter or laser-scanning microscope. It will be apparent to those skilled in the art that excitation/detection means can be augmented by the incorporation of photomultiplier means to enhance detection sensitivity. The differential labels may comprise either two different fluorescent moieties (e.g., fluorescent proteins as described below or the fluorophores rhodamine, fluorescein, SPQ, and others as are known in the art) or a fluorescent moiety and a molecule known to quench its signal.

The fluorescent labels are chosen such that the excitation spectrum of one of the labels (the acceptor label) overlaps with the emission spectrum of the excited fluorescent label (the donor label). The donor label is excited by light of appropriate intensity within the donor's excitation spectrum. The donor then emits some of the absorbed energy as fluorescent light and dissipates some of the energy by FRET to the acceptor fluorescent label. The fluorescent energy it produces is quenched by the acceptor fluorescent label. FRET can be manifested as a reduction in the intensity of the fluorescent signal from the donor, reduction in the lifetime of its excited state, and re-emission of fluorescent light at the longer wavelengths (lower energies) characteristic of the acceptor. When the donor and acceptor labels become spatially separated, FRET is diminished or eliminated.

FRET is commonly performed using a green fluorescent protein and a dye such as a cyanine as the donor and acceptor. Because of its easily detectable green fluorescence, green fluorescent protein (GFP) from the jellyfish Aequorea Victoria has been used. GFP fluorescence does not require a substrate or cofactor; hence, it is possible to use this reporter in numerous species and in a wide variety of cells. Recently, crystallographic structures of wild-type GFP and the mutant GFP S65T reveal that the GFP tertiary structure resembles a barrel (Ormo et al. (1996) Science 273: 1392-1395; Yang, F., Moss, L. G., and Phillips, G. N., Jr. (1996) Nature Biotech 14: 1246-1251). The barrel consists of beta sheets in a compact antiparallel structure. In the center of the barrel; an alpha helix containing the chromophore is shielded by the barrel. The compact structure makes GFP very stable under diverse and/or harsh conditions, such as protease treatment, making GFP an extremely useful reporter in general. A great deal of research is being performed to improve the properties of GFP and to produce GFP reagents useful for a variety of research purposes. New versions of GFP have been developed via mutation, including a “humanized” GFP DNA, the protein product of which has increased synthesis in mammalian cells (see Cormack, et al., (1996) Gene 173, 33-38; Haas, et al., (1996) Current Biology 6, 315-324; and Yang, et al., (1996) Nucleic Acids Research 24, 4592-4593). One such humanized protein is “enhanced green fluorescent protein” (EGFP). Other mutations to GFP have resulted in blue-, cyan- and yellow-fluorescent light emitting versions. These various colored GFPs may be useful in immunoassays of the present invention.

ECIA (enzyme channeling immunoassay; Anal. Biochem. 1056: 223, 1979; Appl. Biochem. Biotechnol. 6, 53-64, 1981) makes use of an antibody and of an analyze tracer each of which carries a different enzyme. The product of the first enzymic reaction constitutes the substrate for the second enzymic reaction. The overall velocity of the two reactions is markedly increased by this co-immobilization.

In SLFIA (substrate-labeled fluorescent immunoassay), an analyte derivative which is labeled with an enzyme substrate competes with the analyte for the binding sites of the anti-analyte antibody. Binding of the substrate-labeled analyte derivative to the antibody prevents the substrate from being reacted enzymically (Burd J. F., Feeney J. E., Carrico R. J., Bogulaski R. C.: Clin. Chem. 23, 1402, 1977; Wong R. C., Burd J. T., Carrico R. J., Buckler R. T., Thoma J., Bogulaski R. C. Clin. Chem. 25, 686, 1979).

If a fluorescent compound is excited in solution with polarized light, the emission which is observed is also polarized. The degree of this polarization depends on the mobility of the excited molecule. The decreasing mobility of a fluorescent tracer when the latter is bound to an antibody is used, in a fluorescence polarization immunoassay, to differentiate between free and bound tracer.

A fluorescence protection immunoassay (H. E. Ullmann: Tokai J. Exp. Clin. Med., Vol. 4, Supplement, pp. 7-32, 1979) is a homogeneous assay which operates in accordance with the competitive method.

In a conventional competitive assay, sufficient anti-analyte antibodies remain free, when analyte concentrations are low, for binding the tracer in such a manner that the label is no longer accessible to an anti-fluorescein antibody and can consequently no longer be quenched. This steric screening can be made even more effective by coupling the anti-analyte antibodies to a sterically demanding component.

In the solid phase antigen technique, the binding of an unwieldy analyte derivative to the tracer antibody prevents, in an analogous manner, it binding simultaneously to the anti-fluorescein antibody.

In a variant of the fluorescence protection immunoassay, the nonspecific absorption of light by active charcoal due to its coupling to the anti-fluorescein antibody is exploited to increase the quenching effect (scavenging effect).

Other techniques have been described, such as, for example, ALFPIA (antigen-labeled fluorescence protection assay; Clin. Chem. 25: 1077, 1979) or SPA (scintillation proximity assay; U.S. Pat. No. 4,569,649; WO 90/11524), in which a signal is generated by means of a radioactive tracer binding close to a scintillator.

In a standard format, the amount of antibodies forming the antibody-antigen complex is directly monitored. This may be accomplished by determining whether labeled anti-xenogenic (e.g., anti-human) antibodies which recognizes an epitope on the antibodies will bind due to complex formation. In a the competitive format, the amount of antibodies in the sample is deduced by monitoring the competitive effect on the binding of a known amount of labeled antibody (or other competing ligand) in the complex.

Antibody-antigen complexes formed are detected by any of a number of known techniques, depending on the format. For example, unlabeled antibodies in the complex may be detected using a conjugate of antixenogeneic Ig complexed with a label, (e.g., an enzyme label). Typically the test sample is incubated with antibodies under conditions that allow the formation of antigen-antibody complexes. Various formats can be employed. For example, a “sandwich assay” may be employed, where antibody bound to a solid support is incubated with the test sample; washed; incubated with a second, labeled antibody to the analyte, and the support is washed again. Analyte is detected by determining if the second antibody is bound to the support. In a competitive format, which can be either heterogeneous or homogeneous, a test sample is usually incubated with antibody and a labeled, competing antigen is also incubated, either sequentially or simultaneously. These and other formats are well known in the art.

Kits suitable for the novel detection system of the instant invention containing the appropriate labeled reagents are constructed by packaging the appropriate materials, including the polypeptides, epitopes or antibodies in suitable containers, along with the remaining reagents and materials required for the conduct of the assay, as well as a suitable set of assay instructions.

The invention is particularly suitable for the screening of novel putative therapeutic agents for activity against the replicating HCV system. Most particularly, libraries of compounds, which may contain tens of compounds to thousands of compounds, may be screened, in order to determine which members of these libraries preferentially allow cell growth and multiplication (that is, are nontoxic) while effectively inhibiting viral replication. In general, this balance is expressed as the therapeutic index, which represents the ratio of the EC₅₀ value to kill the virus divided by the TC₅₀ value to kill the cells. These screening methods are known by those of skill in the art. Generally, the anti-viral agents are tested at a variety of concentrations, for their effect on preventing viral replication in cell culture systems which support viral replication, and then for an inhibition of infectivity or of viral pathogenicity (and a low level of toxicity) in an animal model system.

Previously, quantification of the therapeutic index in the replicon system involved the physical counting of cells which were colored, or other methods which would not support a high throughput format. There the assay operator was required, well-by-well, in an assay plate, which commonly contains 96 wells, to perform such quantifications under a microscope. The reading of a single assay could take hours. In general, there has been a trend in cellular-based assays to move from 96-well plates to 384-well plates, and even to 1096-well plates. In these larger format content plates, quantification well by well by the assay operator is essentially impossible. The method of the invention therefore enables this quantification to be performed by a standard plate reader in an automated manner. This therefore allows assays to be performed in a true high throughput (HTS) setting.

However, even in a low-throughput setting, the methods and compositions taught herein are useful for screening of antiviral agents in that they provide an alternative, and more sensitive means, for detecting the agent's effect on viral replication than existing quantification methods. Moreover, these techniques are particularly useful in cases where the HCV may be able to replicate in a cell line without causing cell death.

The test compounds which may be tested for efficacy by these methods can be obtained using any of the numerous approaches in combinatorial library methods known in the art, including: biological libraries, peptide libraries (libraries of molecules having the functionalities of peptides, but with a novel, non-peptide backbone which are resistant to enzymatic degradation but which nevertheless remain bioactive). (See, e.g., Zuckerman, R. N., et al., J. Med. Chem. 1994, 37: 2678-85); spatially addressable parallel solid phase or solution phase libraries; synthetic library methods requiring deconvolution; the “one-bead one compound” library method; and synthetic library methods using affinity chromatography selection. Methods for the synthesis of molecular and chemical libraries are well known in the art. Other test compounds of interest include, but are not limited to, those which interact with virion components and/or cellular components which are necessary for the binding and/or replication of the virus. Typical anti-viral agents may include, for example, inhibitors of virion polymerase and/or protease(s) necessary for cleavage of the precursor polypeptides. Other test compounds may include those which act with nucleic acids to prevent viral replication, for example, antisense polynucleotides, etc.

Helioxanthin, which is utilized in one of the examples herein, is a natural product which has high activity against Hepatitis B virus (HBV), Hepatitis C virus (HCV), Yellow Fever virus, Dengue Virus, Japanese Encephalitis, West Nile virus and related flaviviruses. Helioxanthin shows potent inhibition of the replication of the virus (viral growth) in combination with very low toxicity to the host cells (see WO0010991 A1). Various derivatives of helioxanthin (see for example U.S. Pat. No. 6,340,704) may also find utility as antiviral agents in high throughput screening in the present assay system.

Other types of putative drugs may be based upon polynucleotides which “mimic” important control regions of the HCV genome, and which may be therapeutic due to their interactions with key components of the system responsible for viral infectivity or replication.

It should be noted that in some circumstances indicator genes other than neomycin resistance might be preferred. Here “Indicator or indicator gene” refers to a nucleic acid encoding a protein, DNA or RNA structure that either directly or through a reaction gives rise to a measurable or noticeable aspect, e.g. a color or light of a measurable wavelength or in the case of DNA or RNA used as an indicator a change or generation of a specific DNA or RNA structure. Examples of an indicator gene is the E. coli lac Z gene which encodes beta-galactosidase, the luc gene which encodes luciferase either from, for example the firefly or Renilla reniformis (the sea pansy), the E. coli phoA gene which encodes alkaline phosphatase, green fluorescent protein and the bacterial CAT gene which encodes chloramphenicol acetyltransferase. Additional preferred examples of an indicator gene are secreted proteins or cell surface proteins that are readily measured by assay, such as radioimmunoassay (RIA), or fluorescent activated cell sorting (FACS), including, for example, growth factors, cytokines and cell surface antigens (e.g. growth hormone, Il-2 or CD4, respectively). “Indicator gene” is understood to also include a selection gene, also referred to as a selectable marker. Examples of suitable selectable markers for mammalian cells are dihydrofolate reductase (DHFR), thymidine kinase, hygromycin, or zeocin.

One of ordinary skill may readily modify the above teachings and use standard isolation techniques to isolate virtually any natural product pursuant to the present invention. Preferred embodiments of the above-described general method may be readily gleaned from the preceding detailed description of the invention and the examples which follow. Having generally described the invention, reference is now made to the following examples which are intended to illustrate preferred embodiments and comparisons but which are not to be construed as limiting the scope of this invention as is more broadly set forth above and in the appended claims.

### EXAMPLE 1

Evaluation of Antiviral Activity of Compounds Against HCV using HCV Replicon-Containing Cells

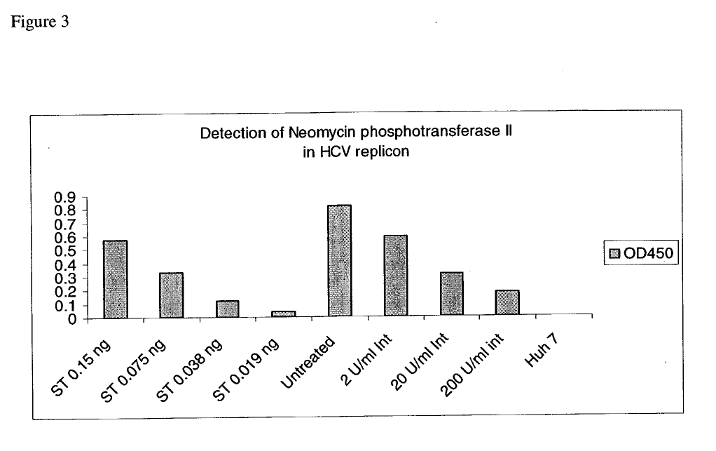

HCV replicon-containing cells were treated with different concentrations of interferon alpha that is known to inhibit viral replication of HCV and HCV replicon. The NPT level of each interferon-treated sample was measured using a captured ELISA. The experiment was carried out as follows; three days after interferon treatment, the cells were lysed and the cell lysate was added to 96 well Maxisorp plate coated with anti-neomycin phosphotransferase II antibody. The plate was incubated at room temperature (25° C. to 28° C.) for 3 hours to allow the binding of NPT in cell lysate to plate-bound anti-NPT. The plate was then washed 6 times with 1× Phosphate Buffered Saline (PBS, from GIBCO, 10010-023). After washing out the unbound proteins, biotin conjugated anti-NPT was used to bind the captured NPT from cell lysate. The complex was then detected with HRP-streptavidin conjugate which binds biotin conjugated anti-NPT. The results of this example are illustrated in FIG. 3.

HCV replicon cells were treated with interferon alpha at the concentration as indicated in each lane. Lanes ST 0.15 ng, ST 0.075 ng, ST 0.038 ng and ST 0.019 ng represent 0.15 ng, 0.075 ng, 0.038 ng and 0.019 ng NPT protein respectively as a standard. Huh 7 represents parental cell line for HCV replicon that contains HCV replicon. Substrate 3,3′,5,5′Tetramethylbenzidine (TMB) was used in this experiment. The reaction was terminated with 0.1 N sulfuric acid and was quantified at OD 450 nm with the aid of a Molecular Devices plate reader.

### EXAMPLE 2

Evaluation of Toxicity of Compounds Towards HCV Replicon-Containing Cells

Cellular protein albumin was used as a marker to monitor cytotoxicity and protein level to provide normalization reference for antiviral activity of compounds.

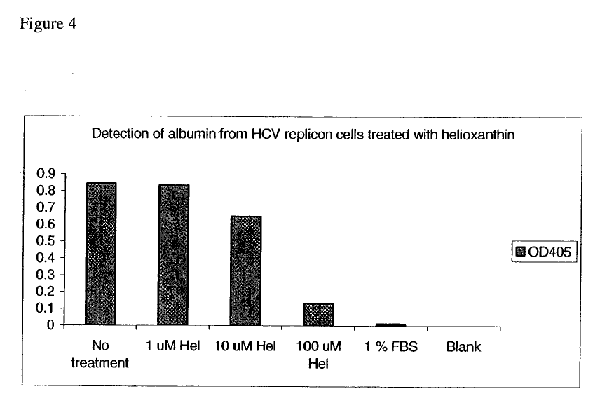

HCV replicon cells were treated for three days with varying concentrations of helioxanthin that is known to have cytotoxicity effect at high concentration. The cells were lysed and the cell lysate was used to bind the plate-bound goat anti-albumin antibody at room temperature (25° C. to 28° C.) for 3 hours. The plate was then washed 6 times with 1× PBS. After washing out the unbound proteins, the mouse monoclonal anti-human serum albumin was applied to bind the albumin on the plate. The complex was then detected using phosphatase-labeled anti-mouse IgG. The results of this experiment are illustrated in FIG. 4.

HCV replicon cells were treated with helioxanthin as indicated. 1% Fetal Bovine Serum (FBS) was used as control to ensure that anti-human albumin antibody has no significant cross reaction with FBS which is a component of the medium for HCV replicon cells. The substrate pNPP was used. The reaction was read at OD 405 nm.

### EXAMPLE 3

Comparison with MTS Assay

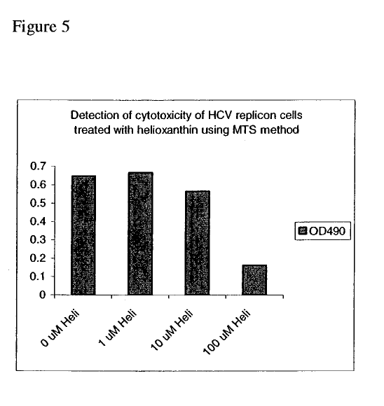

HCV replicon cells were treated with varying concentrations of helioxanthin for three days. Before cells were lysed for detection of albumin as described above, the MTS reagent (Promega, G3580) was added according to manufacturer's instruction to each well, and incubated at 37° C. and read at OD 490 nm. The results are illustrated in FIG. 5.

### EXAMPLE 4

Dot Blot Hybridization Assay

This example describes an improved dot blot assay wherein it was not necessary to purify the HCV RNA.

HCV replicon cells were counted and resuspended in DMEM without phenol red supplemented with L-glutamine, and 10% FBS to yield a cell density of 1>10^{S} cells/ml. Cells were then plated in certain wells of a 96-well flat bottom BioCoat plate in a volume of 100 μL per well. Complete DMEM without phenol red was added to the 36 exterior wells in a volume of 200 μL per well. Plates were incubated overnight at 37° C. in a humidified CO₂ incubator to allow the cells to adhere.

The following day, drug dilutions were prepared in microtiter tubes as follows: (a) drug stock was diluted in complete DMEM to the desired high concentration in a volume of 500 μL; (b) complete DMEM (450 μL) was added to two additional tubes; (c) 10-fold serial dilutions were prepared by adding 50 μL from the previous tube into the next tube. Drug dilutions were then added to the appropriate wells of the microtiter plate in a volume of 100 μL per well. Each dilution was set up in triplicate. Complete medium containing no compound was added to virus control wells in a volume of 100 μL per well. Plates were returned to the incubator and incubated for three days.

On day four, plates were removed from the incubator. 10 μL of MTS solution was added to certain wells of the plate. Plates were placed in the incubator and incubated for two hours. OD 490 was measured on a plate reader and recorded. After MTS staining, the staining solution was discarded completely and the cells were lysed for the RNA dot hybridization experiment.

RLN cell lysis buffer (50 mM Tris-cl, pH 8.0, 140 mM NaCl, 1.5 mM MgCl₂, 0.5% Nonidet P-40 (just before use, 1000 U/ml Rnasin and 1 mM DTT was added) was prepared and precooled to 4° C. After the MTS was removed and discarded, 70 μL of RLN was added to the wells and the plate was incubated for five minutes on ice for a complete cell lysis. After peptiting several times to detach cellular debris from the plate, the total lysate was transferred to a new U bottom 96-well plate. The U bottom plate was placed on a plateholder and subjected to centrifugation of 300 g for 3 minutes. 50 μL of supernatant was carefully removed without touching the nuclear pellet and loaded directly onto manifold wells equipped with Nylon membrane.

Plasmid pHCV1b_NS5.1 (1 μ/μL) was constructed by inserting a PCR fragment amplified from HCV1b replicon I377/NS3-3¹ (genebank accession number is AJ242652) in pGEMT-Easy vector (promega). The PCR fragment included HCV genotype 1b NS5A and 5B (from nt5538 to nt7794). A plus-strand specific probe was made by linearizing the plasmid with Xho I (nt 5570 in HCV 5A region). In vitro transcription with SP6 RNA polymerase produced RNA with approximately 2.2 kb in size.

The size of pHCV1b_NS5.1 should be 3018(vector)+2254(insert)=5272 bp.

The following was added to a sterile Eppendorf tube: pHCV1b-N55 plasmid DNA (1 μg/μl)—10 μl; 10×Restriction buffer#2—6 μl; 100×BSA—0.6 μl; Restriction enzyme Xho I (20 units/μl)—3 μl; Rnase, Dnase-free water—40 μl.

The tube was spun on microfuge to collect everything on the bottom and the reaction mixture was added. The tube was incubated on 37° C. heater blocker for 2 hours. To check if the reaction was complete, 2 μl of reaction solution was removed and mixed with 5 μl of loading buffer for agarose gel. The sample was loaded on a 1% agarose gel with the presence of DNA molecular markers. The complete digestion should give a single DNA band of 5.2 kb in size.

Before in vitro transcription, XhoI digested plasmid was isolated with a QIAquick PCR purification kit to remove the enzyme and other unnecessary components. 500 μl of buffer PB and 10 μl of 3 M NaAc, pH 5.2 was added to the digestion mixture and mixed. The sample was applied to the QIAquick column and centrifuged 30-60 seconds to bind DNA. The flow-through was discovered. QIAquick column was placed back into the collection tube. To wash, 750 μl Buffer PE was added to the QIAquick column and spun. To remove the residual ethanol from Buffer PE, the flow-through was discarded and the QIAquick column was centrifuged for an additional 1 minute as maximum speed. The QIAquick column was placed in a clean 1.5 ml microfuge tube. DNA was eluted by adding 30 μl Buffer EB (10 mM Tris.Cl, pH 8.5) to the center of the QIAquick membrane, letting the column stand for 1 minute, and centrifuging. The final concentration of pHCV1b_NS5.1 was around 1 μg/3 μl. The DNA was stored at −20° C.

This procedure is for the preparation of a radiolabeled hybridization probe generated by transcription of a cloned segment of DNA using the SP6 RNA polymerase promoter in the plasmid. The plasmid should be linearized before the labeling by a restriction digestion. The particular enzyme used must leave either a blunt end or a 5′ overhand, since a 3′ overhand will act as a promoter for transcription.

The length of the RNA transcript will be determined by the concentration of limiting nucleotide, usually UTP. When no carrier UTP is added to the reaction, only a few hundred nucleotides will be synthesized before the reaction runs out of substrate. Therefore, for a probe that was more representative of the genome, carrier UTP was added to 25 μM, for a final UTP concentration of about 30-35 μM. The sequences adjacent to the promoter are to be transcribed, no extra UTP is required. 2 μl 10× RNA polymerase buffer, 0.2 μl 100× BSA, 0.5 μl Rnasin (40 μl/μl promega), 4 μl rNTP with UTP, 3 μl linearized plasmid DNA (pHCV1b-N55, 1 μg/3 μl), 1 μl SP6 RNA polymerase, 5× NTP, 2.5 mM each ATP,GTP,CTP, and 0.1 mM UTP (optional) were added to an Eppendorf tube containing 100 μCi ³²P-UTP (800 Ci/mmole, PerkinElmer life sciences Blu007×) in a volume of 10 μl.

The mixture was incubated at 40° C. for 2 hours, then 4 μl of tRNA carrier (25 μg/μl and 1 μl RQ DNAase (Promega) was added. This mixture was then incubated for five minutes at 37° C.

The reaction was stopped by the addition of 50 μl “stop buffer” containing 10 mM tris·HCl, pH 7-8, 10 mM EDTA, pH 7-8, 150 mM NaCl, and 0.5% SDS.

The RNA was precipitated by adding 170 μl absolute ethanol at room temperature followed by vortexing and microfuging immediately for 5 minutes.

The supernatant was pipetted into an Eppendorf tube for counting and disposal. The radioactivity in the pellet was compared with that in the supernatant tube using the lab survey meter for a rough approximation of the percent of isotope incorporated. Radioactivity in the pellet was 2-3 times that in the supernatant.

The radioactive probate was dissolved in 100 μl water for diluting into the hybridization mix. Membrane was pre-hybridized with the solution containing 500 μg/ml of salmon sperm DNA at 65° C. for at least two hours. The pre-hybridization solution was removed. For hybridization, membrane was incubated with the solution containing HCV RNA probe at 58° C. overnight. (To decrease the non-specific background caused by huge amount of rRNA, hybridization temperature was kept at 58° C. at least.) Membrane was washed with 0.1×SSC and 0.1% SDS for 2 hours at room temperature, then washed with the same washing solution at 65° C. for 1 hour. (The washing solution was changed every half hour.) The membrane was baked at 65° C. for 20 minutes and counted.

### EXAMPLE 5

In Situ High Throughput HCV Replicon Assay

HCV replicon cells were rinsed with PBS once and 2 mls of trypsin was added. Cells were incubated in 37° C. CO₂ incubator for 3-5 minutes. 10 mls of complete medium was added to stop the reaction. Cells were blown gently, put into a 15 ml tube and spun at 1200 rpm for four minutes. The trypsin/medium solution was removed and 5 mls of medium (500 ml DMEM (high glucose)) from BRL catalog #12430-054; 50 mls 10% FBS, 5% Geneticin G418 (50 mg/ml, BRL 10131-035), 5 ml MEM non-essential amino acid (100× BRL #11140-050) and 5 ml pen-strep (BRL #15140-148) was added. Mix carefully.

Cells were plated with screening medium (500 ml DMEM (BRL #21063-029), 50 ml FBS (BRL #10082-147) and 5 mm MEM non-essential amino acid (BRL #11140-050) at 6000-7500 cells/100 μl/well of 96 well plate (6-7.5×10⁵ cells/10 ml/plate). Plates were placed into 37° C. CO₂ incubator overnight.

The following morning, test compounds were diluted into 96 well U bottom plates with media or DMSO/media. 100 μl of the test compound dilution was placed in certain wells of the 96 well plate containing the HCV replicon cells. The plate was incubated at 37° C. in a humidified 5% CO₂ environment for three days.

On day four, the NTPII assay was performed according to the following protocol. The medium was dumped from the plate and the plate was washed once in 200 μl of PBS. The PBS was then dumped and the plate tapped in a paper towel to remove any remaining PBS. Cells were then fixed in situ with 100 μl/well of pre-cooled (−20° C.) methanol: acetone (1:1) and the plates were placed at −20° C. for 30 minutes.

The fixing solution was dumped from the plates and the plates were air-dried completely (approximately one hour). The appearance of the dried cell layer was recorded and the toxic wells were scored with the naked eye by scoring the density of the cells in the well. Cell viability was also determined by CellTites 96® Aqueous One Solution Cell Proliferation Assay (Progema). The assay is a calorimetric method for determining the number of viable cells. In this method, before fixing the cells, 10-20 μl MTS was added to each well according to manufacturer's instruction, incubated at 37° C. and read at OD 490 nm.

The wells were blocked with 200 μl of blocking solution (10% FBS; 3% NGS in PBS) for 30 minutes at room temperature. The blocking solution was removed and 100 μl of rabbit anti-NPTII diluted 1:1000 in blocking solution was added to each well. The plate was then incubated 45 minutes to one hour at room temperature. After incubation, wells were washed six times with PBS-0.05% Tween-20 solution. 100 μl of 1:15,000 diluted En-conjugated goat anti-rabbit in blocking buffer was added to each well and incubated at room temperature for 30-45 minutes. The plate was washed again and 100 μl of enhancement solution (Perkin Elmer #4001-0010) was added to each well. The plate was shaken in a plate shaker three minutes. 95 μl was transferred from each well to a black plate which was read in Victor plate reader—Eu—Lance.

### EXAMPLE 6

Variation of In Situ High Throughput HCV Replicon Assay

It was also possible simultaneously to measure cell viability using an antibody against albumin and the level of NPTII in a single well.

In this method, after the cells were prepared, fixed, and blocked, as described in Example 5 above, blocking buffer was used to dilute rabbit anti-NPTII (1:750) and mouse anti-albumin (1:500), e.g., 10 ml of blocking buffer+10 μl of anti-NPTII+20 μl of anti-albumin. After removing blocking buffer from the plate, 100 μl of diluted primary antibodies was added to each well and allowed to incubate as described in Example 5 above.

Blocking buffer was used to dilute Eu-goat anti-rabbit (1:20,000) and FITC-goat anti-mouse (1:50), e.g., 10 ml of blocking buffer+0.67 μl of Eu-goat anti-rabbit+200 μl of FITC-goat anti-mouse. The plate was washed as described in Example 5 above. 100 μl of diluted secondary antibodies was added and incubated as described in Example 5 above. When the plate was dry, it was read in a Victor plate reader.

It is to be understood by those skilled in the art that the foregoing description and examples are illustrative of practicing the present invention, but are in no way limiting. Variations of the detail presented herein may be made without departing from the spirit and scope of the present invention as defined by the following claims.

What is claimed is:

1. A method for analysis of the antiviral and cell toxicity activity upon Hepatitis C virus of test compound(s) comprising the steps of:
a. constructing a Hepatitis C replicon system containing a gene required for said replicon to replicate (replicon);
b. transfecting and propagating cells which contain said replicon; selecting stably transfected clones;
c. growing and propagating said stably transfected clones; plating said cells and adding test compound(s) to wells containing the cells transfected with the replicon;
d. incubating the transfected cells in the presence of the test compound(s); quantifying a gene product protein produced by the replicon during the incubation period as a measure of antiviral activity; and
e. quantifying a marker for cellular protein expression level produced by the cells during the incubation period as a measure of test compound toxicity.

2. The method of claim 1 wherein the gene required for said replicon to replicate is selected from the group consisting of NPTII, hygromycin B, puromycin, HCV Ns2 protein, HCV Ns3 protein, HCV Ns4a protein, HCV Ns4b protein, HCV Ns5a protein and HCV Ns5b protein.

3. The method of claim 1 wherein said marker is selected from the group consisting of albumin and GADPH.

4. The method of claim 2 wherein said marker is selected from the group selected from albumin and GADPH.

5. The method of claim 2 wherein said gene is NPTII.

6. The method of claim 5 wherein said marker is albumin.

7. The method of claim 1 wherein the amount of gene product protein produced during the incubation period is measured by an assay selected from the group consisting of a luminescence assay, a chemiluminescence assay, an enzyme-multiplied immunoassay technology (EMIT) assay, a fluorescence resonance excitation transfer immunoassay (FRET assay, an enzyme channeling immunoassay (ECIA) assay, a substrate-labeled fluorescent immunoassay (SLFIA) assay, a fluorescence polarization assay, a fluorescence protection assay, an antigen-labeled fluorescence protection assay (ALFPIA), or a scintillation proximity assay (SPA).

8. The method of claim 1 wherein the amount of the marker for cellular protein expression level is measured by visually inspecting the wells after the incubation period.

9. The method of claim 1 wherein the amount of the marker for cellular protein expression level is measured using an MTS uptake assay.

10. The method of claim 1 wherein a kit suitable for use in the high throughput format assay is prepared, wherein said kit containing the appropriate labeled reagents is constructed by packaging the appropriate materials, including the polypeptides, epitopes or antibodies in suitable containers, along with the remaining reagents and materials required for the conduct of the assay, as well as a suitable set of assay instructions.

11. A method for simultaneous analysis of the antiviral and cell toxicity activity upon Hepatitis C virus of test compound(s) comprising the steps of:
a. constructing a Hepatitis C replicon system containing a gene required for said replicon to replicate (replicon);
b. transfecting and propagating cells which contain said replicon;
c. selecting stably transfected clones;
d. growing and propagating said stably transfected clones;
e. plating said cells and adding test compound(s) to wells containing the cells transfected with the replicon;
f. incubating the transfected cells in the presence of the test compound(s);
g. simultaneously quantifying the amount of a gene product protein produced by the replicon during the incubation period and the amount of a marker for cellular protein expression level produced by the cells during the incubation period.

12. The method of claim 11 wherein the gene required for said replicon to replicate is selected from the group consisting of NPTII, hygromycin B, puromycin, HCV Ns2 protein, HCV Ns3 protein, HCV Ns4a protein, HCV Ns4b protein, HCV Ns5a protein and HCV Ns5b protein.

13. The method of claim 11 wherein said marker is selected from the group consisting of albumin and GADPH.

14. The method of claim 12 wherein said marker is selected from the group consisting of albumin and GADPH.

15. The method of claim 12 wherein said gene is NPTII.

16. The method of claim 15 wherein said marker is albumin.

17. The method of claim 11 wherein the amount of gene product protein produced during the incubation period is measured by an assay selected from the group consisting of a luminescence assay, a chemiluminescence assay, an enzyme-multiplied immunoassay technology (EMIT) assay, a fluorescence resonance excitation transfer immunoassay (FRET assay, an enzyme channeling immunoassay (ECIA) assay, a substrate-labeled fluorescent immunoassay (SLFIA) assay, a fluorescence polarization assay, a fluorescence protection assay, an antigen-labeled fluorescence protection assay (ALFPIA), or a scintillation proximity assay (SPA).

18. The method of claim 11 wherein the amount of the marker for cellular protein expression level is measured by visually inspecting the wells after the incubation period.

19. The method of claim 11 wherein the amount of the marker for cellular protein expression level is measured using an MTS uptake assay.

20. The method of claim 11 wherein a kit suitable for use in the high throughput format assay is prepared, wherein said kit containing the appropriate labeled reagents is constructed by packaging the appropriate materials, including the polypeptides, epitopes or antibodies in suitable containers, along with the remaining reagents and materials required for the conduct of the assay, as well as a suitable set of assay instructions.
